SEQUENCE LISTING (6 sequences)

SEQ ID NO: 1 - protein, 575 aa, Homo sapiens
MWSLLLCGLSIALPLSVTADGCKDIFMKNEILSASQPFAFNCTFPPITSGEVSVTWYKNSSKIPVSKIIQSRIHQDETWILFLPMEWGDSGVYQCVIKGRDSCHRIHVNLTVFEKHWCDTSIGGLPNLSDEYKQILHLGKDDSLTCHLHFPKSCVLGPIKWYKDCNEIKGERFTVLETRLLVSNVSAEDRGNYACQAILTHSGKQYEVLNGITVSITERAGYGGSVPKIIYPKNHSIEVQLGTTLIVDCNVTDTKDNTNLRCWRVNNTLVDDYYDESKRIREGVETHVSFREHNLYTVNITFLEVKMEDYGLPFMCHAGVSTAYIILQLPAPDFRAYLIGGLIALVAVAVSVVYIYNIFKIDIVLWYRSAFHSTETIVDGKLYDAYVLYPKPHKESQRHAVDALVLNILPEVLERQCGYKLFIFGRDEFPGQAVANVIDENVKLCRRLIVIVVPESLGFGLLKNLSEEQIAVYSALIQDGMKVILIELEKIEDYTVMPESIQYIKQKHGAIRWHGDFTEQSQCMKTKFWKTVRYHMPPRRCRPFPPVQLLQHTPCYRTAGPELGSRRKKCTLTTG

SEQ ID NO: 2 - protein, 6 aa, Homo sapiens
MKNEIL

SEQ ID NO: 3 - protein, 15 aa, Homo sapiens
EKHWCDTSIGGLPNL

SEQ ID NO: 4 - protein, 7 aa, Homo sapiens
YKQILHL

SEQ ID NO: 5 - protein, 6 aa, Homo sapiens
IKGERF

SEQ ID NO: 6 - protein, 10 aa, Homo sapiens
QAILTHSGKQ

(19) United States
(12) Patent Application Publication
(10) Pub. No.: US 2025/0282859 A1
(43) Pub. Date: Sep. 11, 2025

(54) USE OF IL-36 INHIBITORS FOR THE TREATMENT OF NETHERTON SYNDROME

(71) Applicants: INSTITUT NATIONAL DE LA SANTÉ ET DE LA RECHERCHE MÉDICALE, Paris (FR); FONDATION IMAGINE, Paris (FR); ASSISTANCE PUBLIQUE-HÔPITAUX DE PARIS (APHP), Paris (FR); UNIVERSITÉ PARIS CITÉ, Paris (FR)

(72) Inventors: Alain HOVNANIAN, Paris (FR); Olivier GOUIN, Romainville (FR); Claire BARBIEUX, Esch-sur-Alzette (LU); Evgeniya PETROVA, Paris (FR)

(21) Appl. No.: 18/571,893

(22) PCT Filed: Jul. 11, 2022
(86) PCT No.: PCT/EP2022/069292

§ 371 Date: Dec. 19, 2023

(30) Foreign Application Priority Data
Jul. 12, 2021 (EP) 21305968.6

(51) Int. Cl. C07K 16/24; A61K 39/00; A61P 17/00; C07K 16/28

(52) U.S. Cl. CPC C07K 16/244; A61P 17/00; C07K 16/2866; A61K 2039/505

(57) ABSTRACT

Netherton syndrome (NS) is a rare recessive skin disorder caused by loss-of-function mutations in SPINK5 encoding the protease inhibitor LEKTI. NS patients present with typical ichthyosis linearis circumflexa (NS-ILC) or scaly erythroderma (NS-SE). Here the inventors employed a combination of several molecular profiling methods to comprehensively characterize the skin, immune cells and allergic phenotypes of NS-ILC and NS-SE patients. In particular, they studied a cohort of 13 NS patients comprising 9 NS-ILC and 4 NS-SE. Integrated multi-omics revealed abnormal epidermal proliferation and differentiation and IL-17/IL-36 signatures in lesion skin and in blood in both NS endotypes. This study thus identifies IL-17/IL-36 as predominant signaling axes in both NS endotypes and unveils molecular features distinguishing NS-ILC and NS-SE. In particular, blocking of IL36 signaling would therefore represent a novel therapeutic strategy for NS, in particular in NS-SE patients.

This application includes as the Sequence Listing the complete contents of the xml file “11450859us_sequence_listing”, created Nov. 22, 2024, containing 6,867 bytes, hereby incorporated by reference.

### FIELD OF THE INVENTION

The present invention is in the field of medicine, in particular dermatology.

### BACKGROUND OF THE INVENTION

Netherton Syndrome (NS) (OMIM 256500) is a rare and severe recessive genetic skin disease characterized by the diagnostic triad of ichthyosiform erythroderma, a specific hair shaft abnormality known as trichorrhexis invaginata and high serum IgE levels with atopic manifestations. NS is an orphan disease with currently no satisfactory treatment. NS is caused by recessive loss of function mutations in SPINK5 (1), encoding the serine protease inhibitor LEKTI expressed at the granular layer-stratum corneum interface (2, 3), leading to unopposed activity of cutaneous proteases, including kallikrein-related proteinases (KLK) 5, KLK7, and KLK14 and epidermal elastase 2 (4-8). Unrestrained epidermal protease activity causes excessive desquamation resulting in a profound skin barrier defect and triggers the activation of inflammatory pathways (9-12). NS patients display increased transepidermal water loss, superficial scaling, and redness of the skin. At birth or shortly thereafter, NS patients present with diffuse erythroderma and scaling. During childhood and adulthood, cutaneous lesions evolve in some patients towards ichthyosis linearis circumflexa (ILC), which refers to fluctuating polycyclic and serpiginous erythematous lesions bordered by a double collar of scales, while other patients retain a scaly erythrodermic phenotype (SE). Both NS-ILC and NS-SE patients usually develop increased allergen-specific serum IgE with associated multiple airborne and/or food allergies. The presence of immune system abnormalities remains debated (13, 14).

Histological analyses of NS skin shows stratum corneum detachment, parakeratosis, reduced granular layer (15) with epidermal hyperplasia and hyper-papillomatosis with increased expression of proliferation markers such as keratin 16 (KRT16) (16). Differentiation markers such as involucrin (IVL), loricrin (LOR), and filaggrin (FLG) are diffuse in NS upper epidermal layers with a reduction of desmosomal components (5), which contribute to the skin barrier defect. Several studies pointed to an altered lipid composition of NS stratum corneum (17, 18), which is likely to aggravate the skin barrier defect and inflammation. Epidermal hyperplasia, abnormal differentiation, and altered lipid composition are features in common with other inflammatory skin diseases, including atopic dermatitis (AD), psoriasis and ichthyoses. A transcriptomic study comparing different forms of ichthyoses including NS, with psoriasis and AD, revealed that NS transcriptomic signature was closer to psoriasis, although psoriatic patients lack allergic manifestations (16).

In addition to skin barrier disruption, epidermal proteases such as KLK5 and KLK14 play a role in skin inflammation by activating Protease-activated receptor 2 (PAR2), which in turn triggers the expression of pro-inflammatory cytokines such as TSLP, CXCL8 and TNFa (9, 19-21). Bacterial- and allergen-derived proteases can also induce PAR2 signaling and activate downstream pro-inflammation pathways. In particular, NS patients are susceptible to infections by Staphylococcus aureus, which also induces an immune response driven by the Th17 and IL-36 axis, thus enhancing skin inflammation (22, 23). Previous transcriptomic analyses of NS lesion skin showed an IL-17 signature with increased expression of target genes encoding anti-microbial peptides or IL-17-related cytokines (16, 24). NS patients also displayed an enrichment in circulating lymphocytes expressing IL-17 and IL-22 (25), suggesting that NS could be a Th17-driven disorder resulting from skin barrier impairment (14). The involvement of the Th17 axis in NS pathogenesis is supported by the successful treatment of NS patients with anti-IL-17A antibodies (26-28). Although anti-IL-17A therapy results in a significant clinical benefit, it was recently reported that improvement could not be durably sustained, suggesting that other biological pathways are likely to contribute to NS pathogenesis.

### SUMMARY OF THE INVENTION

The present invention is defined by the claims. In particular, the present invention relates to the use of IL-36 inhibitors for the treatment of Netherton syndrome.

### DETAILED DESCRIPTION OF THE INVENTION

Netherton syndrome (NS) is a rare recessive skin disorder caused by loss-of-function mutations in SPINK5 encoding the protease inhibitor LEKTI. NS patients suffer from a severe skin barrier defect, display inflammatory skin lesions and superficial scaling with atopic manifestations. They present with typical ichthyosis linearis circumflexa (NS-ILC) or scaly erythroderma (NS-SE). Here the inventors employed a combination of several molecular profiling methods to comprehensively characterize the skin, immune cells and allergic phenotypes of NS-ILC and NS-SE patients. In particular, they studied a cohort of 13 NS patients comprising 9 NS-ILC and 4 NS-SE. Integrated multi-omics revealed abnormal epidermal proliferation and differentiation and IL-17/IL-36 signatures in lesion skin and in blood in both NS endotypes. While the molecular profiles of NS-ILC and NS-SE lesion skin were very similar, non-lesion skin of each disease subtype displayed distinctive molecular features. Non-lesion and lesion NS-SE epidermis showed activation of the type I IFN signaling pathway, while lesion NS-ILC skin differed from non-lesion NS-ILC skin by increased complement activation and neutrophil infiltration. Serum cytokine profiling and immunophenotyping of circulating lymphocytes showed a Th2-driven allergic response in NS-ILC, whereas NS-SE patients displayed mainly a Th9 axis with increased CCL22/MDC and CCL17/TARC serum levels. This study identifies IL-17/IL-36 as predominant signaling axes in both NS endotypes and unveils molecular features distinguishing NS-ILC and NS-SE. In particular, blocking of IL36 signaling would therefore represent a novel therapeutic strategy for NS.

Accordingly, the present invention relates to a method of treating Netherton syndrome in a patient in need thereof comprising administering to the patient a therapeutically effective amount of an IL-36 inhibitor.

As used herein, the term “Netherton syndrome” or “NS” has its general meaning in the art and refers to a rare and severe autosomal recessive skin disorder characterized by congenital erythroderma, a specific hair-shaft abnormality, and atopic manifestations with high IgE levels. Generalized scaly erythroderma (SE) is apparent at or soon after birth and usually persists. Scalp hair is sparse and brittle with a characteristic ‘bamboo’ shape under light microscopic examination due to invagination of the distal part of the hair shaft to its proximal part. Atopic manifestations include eczema-like rashes, atopic dermatitis, pruritus, hay fever, angioedema, urticaria, high levels of IgE in the serum, and hypercosinophilia. Life-threatening complications are frequent during the neonatal period, including hypernatremic dehydration, hypothermia, extreme weight loss, bronchopneumonia, and sepsis. During childhood, failure to thrive is common as a result of malnutrition, metabolic disorders, chronic erythroderma, persistent cutaneous infections, or enteropathy.

In some embodiments, the method of the present invention is particularly suitable for the treatment of a patient who retains a scaly erythrodermic phenotype (SE).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “IL-36” refers to human IL-36alpha (UniProtKB Q9UHA7), IL-36beta (UniProtKB Q9NZH7) and or IL-36gamma (UniProtKB Q9NZH8). IL-36 are cytokines that activate NF-kappa-B and MAPK signaling pathways in target cells linked to a pro-inflammatory response. The functional IL-36 receptor named as “IL-36R” is a heterodimer containing interleukin-1 receptor-like 2 (IL1RL2) as the ligand binding moiety and the IL-1 receptor accessory protein (IL1RAP).

Accordingly, as used herein the terms “IL-36 inhibitor” refers to any compound that is able to inhibit the IL-36 signaling pathway. The IL-36 inhibitor to be used in the methods described herein is a molecule that blocks, suppresses, or reduces (including significantly) the biological activity of an IL-36 cytokine, including downstream pathways mediated by IL-36 signaling. Thus the term “IL-36 inhibitor” implies no specific mechanism of biological action whatsoever, and is deemed to expressly include and encompass all possible pharmacological, physiological, and biochemical interactions with an IL-36 cytokine or its receptor whether direct or indirect.

In some embodiments, the IL-36 inhibitor is selected from the group consisting of antibodies directed against an IL-36 cytokine and antibodies directed against the IL-36 receptor (e.g., an antibody specifically binds IL1RL2 or IL1RAP or the dimeric complex formed thereby). More particularly, the antibody of the present invention binds to the extracellular domain of IL1RL2.

As used herein, the term “IL1RL2” refers to the Interleukin-1 receptor-related protein 2. An exemplary amino acid sequence for IL1RL2 is shown as SEQ ID NO:1. The extracellular domain of IL1RL2 typically consists of the amino acid sequence that ranges from the amino acid residue at position 20 to the amino acid residue 335 in SEQ ID NO:1.

>sp|Q9HB29|ILRL2_HUMAN Interleukin-1 receptor-like
2 OS = Homo sapiens OX = 9606 GN = IL1RL2 PE = 1
SV = 2
SEQ ID NO: 1
MWSLLLCGLSIALPLSVTADGCKDIFMKNEILSASQPFAFNCTFPPITSG
EVSVTWYKNSSKIPVSKIIQSRIHQDETWILFLPMEWGDSGVYQCVIKGR
DSCHRIHVNLTVFEKHWCDTSIGGLPNLSDEYKQILHLGKDDSLTCHLHF
PKSCVLGPIKWYKDCNEIKGERFTVLETRLLVSNVSAEDRGNYACQAILT
HSGKQYEVLNGITVSITERAGYGGSVPKIIYPKNHSIEVQLGTTLIVDCN
VIDTKDNTNLRCWRVNNTLVDDYYDESKRIREGVETHVSFREHNLYTVNI
TFLEVKMEDYGLPFMCHAGVSTAYIILQLPAPDFRAYLIGGLIALVAVAV
SVVYIYNIFKIDIVLWYRSAFHSTETIVDGKLYDAYVLYPKPHKESQRHA
VDALVLNILPEVLERQCGYKLFIFGRDEFPGQAVANVIDENVKLCRRLIV
IVVPESLGFGLLKNLSEEQIAVYSALIQDGMKVILIELEKIEDYTVMPES
IQYIKQKHGAIRWHGDFTEQSQCMKTKFWKTVRYHMPPRRCRPFPPVQLL
QHTPCYRTAGPELGSRRKKCTLTTG

In some embodiments, the IL-36 inhibitors is an antibody that binds to the amino acid sequence that ranges from the amino acid residue at position 20 to the amino acid residue 335 in SEQ ID NO: 1. More particularly, the antibody binds to a conformational epitope that comprises at least one following amino acid sequences selected from the group consisting of MKNEIL (SEQ ID NO: 2), EKHWCDTSIGGLPNL (SEQ ID NO:3), YKQILHL (SEQ ID NO:4), IKGERF (SEQ ID NO: 5 and QAILTHSGKQ (SEQ ID NO:6).

As used herein, the term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F (ab′) 2 fragments can be generated by treating the antibody with pepsin. The resulting F (ab′) 2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “Nanobody®”. For a general description of (single) domain antibodies, reference is also made to the prior art cited above, as well as to EP 0 368 684, Ward et al. (Nature 1989 Oct. 12; 341 (6242): 544-6), Holt et al., Trends Biotechnol., 2003, 21 (11): 484-490; and WO 06/030220, WO 06/003388.

As used herein, the term “epitope” refers to a specific arrangement of amino acids located on a protein or proteins to which an antibody binds. Epitopes often consist of a chemically active surface grouping of molecules such as amino acids or sugar side chains, and have specific three dimensional structural characteristics as well as specific charge characteristics. Epitopes can be linear or conformational, i.e., involving two or more sequences of amino acids in various regions of the antigen that may not necessarily be contiguous.

In some embodiments, the antibody is a humanized antibody. As used herein, the term “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans. In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Anti-IL36R antibodies are well known in the art and includes those described in WO2013074569 and WO2016168542. In some embodiments, the IL-36R antibody of the present invention is the antibody disclosed in Ganesan R, Raymond E L, Mennerich D, et al. Generation and functional characterization of anti-human and anti-mouse IL-36R antagonist monoclonal antibodies. MAbs. 2017; 9:1143-1154. In some embodiments, the anti-IL36R antibody of the present invention is Spesolimab.

In some embodiments, the antibody does not comprise an Fc portion that induces antibody dependent cellular cytotoxicity (ADCC). The terms “Fc domain,” “Fc portion,” and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof. Unless otherwise specified, the commonly accepted Kabat amino acid numbering for immunoglobulins is used throughout this disclosure (see Kabat et al. (1991) Sequences of Protein of Immunological Interest, 5th ed., United States Public Health Service, National Institute of Health, Bethesda, MD). In some embodiments, the antibody of the present invention does not comprise an Fc domain capable of substantially binding to a FcgRIIIA (CD16) polypeptide. In some embodiments, the antibody of the present invention lacks an Fc domain (e.g. lacks a CH2 and/or CH3 domain) or comprises an Fc domain of IgG2 or IgG4 isotype. In some embodiments, the antibody of the present invention consists of or comprises a Fab, Fab′, Fab′-SH, F (ab′) 2, Fv, a diabody, single-chain antibody fragment, or a multispecific antibody comprising multiple different antibody fragments. In some embodiments, the antibody of the present invention is not linked to a toxic moiety. In some embodiments, one or more amino acids selected from amino acid residues can be replaced with a different amino acid residue such that the antibody has altered C2q binding and/or reduced or abolished complement dependent cytotoxicity (CDC). This approach is described in further detail in U.S. Pat. Nos. 6,194,551.

In some embodiments, the IL-36 inhibitor is an inhibitor of an IL-36 cytokine expression or of a IL-36 receptor (i.e. IL1RL2 or IL1RAP) expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of IL-36 or IL-36R mRNA by binding thereto and thus preventing protein translation or increasing mRNA degradation, thus decreasing the level of IL-36 or IL-36R, and thus activity, in a cell. For example, antisense oligonucleotides of at least about 15 bases and complementary to unique regions of the mRNA transcript sequence encoding IL-36 or IL-36R can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. IL-36 or IL-36R gene expression can be reduced by contacting a patient or cell with a small double stranded RNA (dsRNA), or a vector or construct causing the production of a small double stranded RNA, such that IL-36 or IL-36R gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing IL-36 or IL-36R. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Typicaly the IL-36 inhibitor of the present invention is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils.

In some embodiments, it may be desirable to administer the IL-36 inhibitor of the present in a topical formulation. As used herein the term “topical formulation” refers to a formulation that may be applied to skin. Topical formulations can be used for both topical and transdermal administration of substances. As used herein, “topical administration” is used in its conventional sense to mean delivery of a substance, such as a therapeutically active agent, to the skin or a localized region of a subject's body. As used herein, “transdermal administration” refers to administration through the skin. Transdermal administration is often applied where systemic delivery of an active is desired, although it may also be useful for delivering an active to tissues underlying the skin with minimal systemic absorption. Typically, the topical pharmaceutically acceptable carrier is any substantially nontoxic carrier conventionally usable for topical administration of pharmaceuticals in which the IL-36 inhibitor of the present invention will remain stable and bioavailable when applied directly to skin surfaces. For example, carriers such as those known in the art effective for penetrating the keratin layer of the skin into the stratum corneum may be useful in delivering the IL-36 inhibitor of the present invention to the area of interest. Such carriers include liposomes. The IL-36 inhibitor of the present invention can also be administered in combination with other pharmaceutically effective agents including, but not limited to, antibiotics, other skin healing agents, and antioxidants. In some embodiments, the topical formulation of the present invention comprises a penetration enhancer. As used herein, “penetration enhancer” refers to an agent that improves the transport of molecules such as an active agent (e.g., a drug) into or through the skin. Various conditions may occur at different sites in the body either in the skin or below creating a need to target delivery of compounds. Thus, a “penetration enhancer” may be used to assist in the delivery of an active agent directly to the skin or underlying tissue or indirectly to the site of the disease or a symptom thereof through systemic distribution. A penetration enhancer may be a pure substance or may comprise a mixture of different chemical entities.

The invention will be further illustrated by the following figures and examples. However, these examples and figures should not be interpreted in any way as limiting the scope of the present invention.

### FIGURES

FIG. 1. Serum levels of proinflammatory IL-36y and CCL20. The levels were measured by Luminex in NS patients (n=13) and healthy controls (n=11). Each dot represents one sample and the lines correspond to the mean±SEM. Differences are statistically significant for p<0.05 (*p<0.05, **p<0.01, ***p<0.001).

### EXAMPLE

### Methods

### Patient Cohort

A cohort of 13 NS patients from 10 kindreds with a median age of 32 years and 19 age-matched HC (median age of 35 years) was recruited. NS was confirmed by the identification of deleterious mutations in SPINK5 and negative LEKTI immunostaining for the 13 NS patients including 9 NS-ILC and 4 NS-SE patients. Demographic information of patients are provided in Table 1. The protocol was approved by the international review board of Necker Hospital (Clinical Trial NCT 020 813 13), and the study was conducted in accordance with the Declaration of Helsinki principles. All patients and HC gave written and informed consent.

### RNA Sequencing Library of NS Skin

Total RNA was isolated from 5 mm skin biopsy. After a DNase treatment with HL-dsDNase (ArcticZymes), complemetary DNA (cDNA) was prepared using the NuGEN Ovation RNA-Seq System from 100 ng of total RNA. RNA-Seq libraries were sequenced on an Illumina HiSeq. Further details are provided in Supplementary Materials. The transcriptomic data were deposited at GEO data repository with the accession number GSE164285.

### Proteomic Analysis of NS Skin

Proteins were extracted from frozen skin samples using RapiGest containing buffer followed by reduction and alkylation. Proteins were digested by first incubating with LysC followed by incubating with Trypsin. Data were acquired with a Q-Exactive Plus (Thermo Scientific, Bremen, Germany) mass spectrometer. Further details are provided in Supplementary Materials. Mass spectrometry data as well as data analysis results have been deposited to ProteomeXchange via MassIVE (ID: PXD023658).

### Statistical Analyses

Statistical analyses were performed with GraphPad Prism v8.4.3 and R software (R Development Core Team, 3.5.0). Differences were considered significant for p<0.05.

### Results

### 1. Patient Characterization

To study the molecular mechanisms involved in NS, we analyzed a cohort of 13 patients, comprising 9 NS-ILC and 4 NS-SE (Table 1). NS diagnosis was confirmed by the identification of deleterious mutations in SPINK5 and negative LEKTI immunostaining for each patient. NS-ILC patients presented with flares of erythematous, serpiginous lesions with a double scaly edge on their trunk and/or limbs. In contrast, NS-SE patients had extensive scaly and red skin and often displayed a lichenified appearance of flexion creases (data not shown).

In both endotypes, histological analyses revealed stratum corneum detachment, psoriasiform epidermal hyperplasia with elongated rete ridges and parakeratosis in non-lesion and lesion skin (data not shown). The stratum granulosum was thicker in non-lesion NS-SE skin and was almost completely absent in lesion skin from both NS endotypes. Subcorneal microabscesses were seen in lesion NS-ILC skin only. Histological observations were supported by immunostaining of skin sections with increased Ki67 and p63 positive cells in the basal and suprabasal layers in lesion skin and to a lesser extent in non-lesion skin, suggesting increased epidermal proliferation. Expression of epidermal regeneration markers KRT16 and KRT6 was also elevated in suprabasal layers of lesionl NS skin and in non-lesion NS-SE skin (data not shown). LOR, IVL and FLG staining was enhanced in non-lesion NS-SE skin and to a lesser extent in NS-ILC, but it was absent or substantially reduced in lesion NS skin (data not shown). Marked inflammatory infiltrates were observed in NS lesion skin in both subtypes. Immunostaining with MPO and tryptase antibodies showed massive neutrophil and mast cell infiltrates in lesion NS-ILC and lesion NS-SE skin, respectively (data not shown). No eosinophils or infiltrating lymphocytes were observed (data not shown).

Total serum IgE levels were similarly increased by 3- to 40-fold in both NS-ILC and NS-SE patients (Table 1) and were associated with several atopic manifestations. Serum component allergen-specific IgE showed high levels of specific IgE against pollens, dust mites, pets and nuts (data not shown). Likewise, all patients analyzed had elevated specific IgE against cross-species allergens such as profilin, tropomyosin or PR10 proteins (data not shown). These results showed that the two clinical subtypes of NS shared common histological and allergic sensitization features with distinct immune cell skin infiltrates.

### 2. Transcriptomic and Proteomic Profiling of NS Skin Shows IL-17 and IL-36 Driven Immune Response in Skin and Peripheral Blood.

To investigate the transcriptomic signature of NS, RNAseq was performed from lesion and non-lesion skin biopsies obtained from NS patients or healthy donors. Principal component analysis (PCA) revealed a clear separation between NS lesion, non-lesion and healthy control samples (data not shown), with non-lesion skin of NS-ILC patients being close to HC and non-lesion NS-SE skin resembling lesion samples. A global analysis was performed to compare HC, NS non-lesion and NS lesion skin (data not shown) and the DEGs identified in each comparison were submitted to DAVID online tool. Epidermal differentiation, immune response, inflammation and proliferation were the most enriched biological processes in DEGs. Lesion and non-lesion skin showed similar features with DEGs mainly involved in epidermal differentiation and immune response when compared to healthy controls. Inflammation and proliferation were specifically enriched in lesion skin (data not shown).

In parallel, proteomic profiling of non-lesion and lesion skin biopsies from the same patients showed a clear separation between NS and healthy controls as revealed at the RNA level (data not shown). RNAseq and proteomic were positively correlated (data not shown) and showed enrichment for the same biological processes. Up-regulated proteins in NS non-lesion and lesion skin (data not shown) were mainly involved in epidermal differentiation, cell adhesion, anti-microbial response and the immune response, whereas multiple proteins crucial in extracellular matrix organization were downregulated compared to healthy skin biopsies (data not shown). Proteins involved in the immune response, especially in the IL-17/IL-36 pathways, were specifically upregulated in lesion skin.

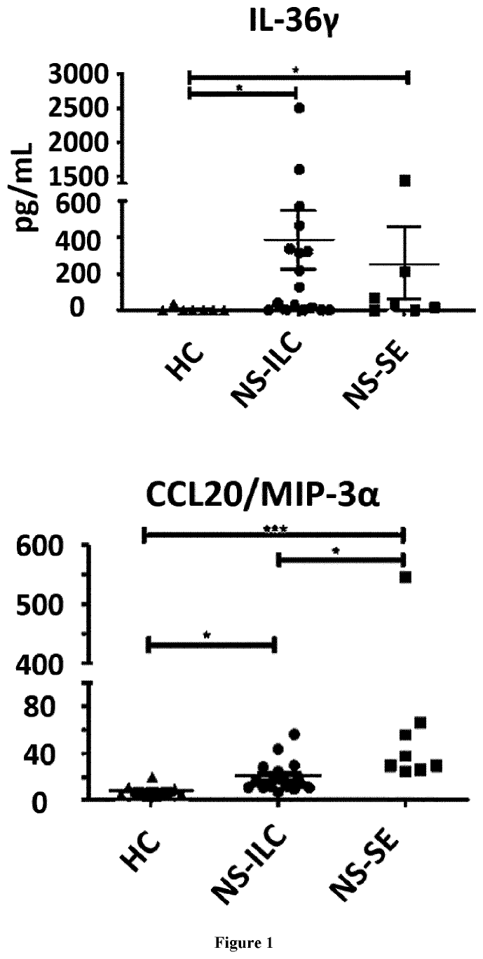

The expression of IL-17- and IL-36-related genes was increased in NS skin (data not shown), consistent with IL-17A and IL-36γ polarization of lesion NS transcriptome (data not shown). Of note, non-lesion NS-SE skin showed enhanced expression of IL-36γ-induced genes. Genes involved in IL-17/IL-36 pathways (S100A7/8/9, IL36G) were among the ones with the highest transcript and protein fold changes (data not shown). Immunostaining of skin sections revealed a significant increase in IL-36γ in lesion and non-lesion NS skin and S100 proteins in lesion NS skin compared to healthy controls (data not shown). Evidence for IL-17 and IL-36 signature was also sought in NS patients sera. IL-36γ and the IL-17-induced chemokine CCL20 serum levels were significantly increased in NS patient compared to HC (FIG. 1). Previous reports have demonstrated a Th17 skewing in NS patients (25), which was confirmed in this patient cohort with a more pronounced skewing in NS-SE than in NS-ILC, although no significant differences were observed (data not shown). Overall, this IL-17/IL-36 transcriptomic and proteomic signature was identified in both NS endotypes and seemed to be more pronouced in NS-SE patients than in NS-ILC.

### Discussion

Here, we describe the global changes in NS-ILC and NS-SE patients compared to HC, at the clinical, histological and molecular levels. In both clinical subtypes, transcriptomic and proteomic signatures of non-lesion skin revealed abnormalities in epidermal proliferation and differentiation. Epidermal hyperproliferation and impaired differentiation are major characteristics of NS lesion skin which have been previously reported (15, 16, 31). Complete loss of differentiation markers seen in lesion NS-SE may result from increased serine protease activity in lesion NS-SE compared to NS-ILC (18), which is supported in our study by downregulation of protease inhibitors in NS-SE patients. Multi-omic profiling showed a prominent IL-17/IL-36 signature in both NS endotypes in non-lesion and lesion skin, which could contribute to epidermal hyperproliferation and impaired differentiation. IL-17 and IL-36 cytokines trigger epidermal proliferation (32, 33) and inhibit epidermal differentiation in psoriasis (34, 35). We previously reported, and confirmed in this study, that lesion NS-ILC skin displayed marked neutrophil infiltrates, whereas lesion NS-SE skin was mainly infiltrated with mast cells (27). Neutrophil gelatinase-associated lipocalin and histamine released by mast cells could participate in the dysregulation of epidermal differentiation in NS-ILC and NS-SE patients, respectively (36, 37). Non-lesion skin from the two endotypes differed, wound healing markers (KRT6 and KRT16) being enhanced in NS-SE compared to NS-ILC, suggesting that skin homeostasis is more disrupted in NS-SE patients. Non-lesion NS-SE skin also displayed increased expression of epidermal differentiation markers compared to NS-ILC, which could result from the enhanced IFN signature in non-lesion NS-SE skin. Indeed, IFN-β is required to induce differentiation of cultured keratinocytes (38).

Multi-omic analyses of NS skin revealed increased immune and inflammatory responses, which mainly relied on IL-17 and IL-36 cytokines. IL-17A and F are secreted by Th17 cells, and IL-17C is expressed by keratinocytes, while IL-36a, B and y cytokines are mainly found in the epidermis (39). Staphylococcus aureus is the most represented strain in NS skin microbiome (22) and contributes to the IL-17/IL-36 signature by disrupting the skin barrier. Therefore, the skin barrier defect is likely to trigger an immune response in NS patients with no evidence for an immunodeficiency as previously suggested (14). Of note, the inflammatory properties of IL-17 and IL-36 pathways are enhanced by their capacity to mutually regulate each other (40-42). Recent studies have shown that IL-36 cytokines are the most upstream mediator of skin inflammation in murine models of psoriasis (43-45). In keratinocytes, IL-17 and IL-36 cytokines induce CCL20 and CXCL8 expression and secretion, which contribute to the inflammatory environment by recruiting neutrophils (46, 47). In psoriatic patients, IL-17A/F and IL-36α and γ cytokines have been shown to be crucial in the course of the disease and to correlate with disease severity (48, 49). Recently, a transcriptomic study showed increased expression of IL-36x and y cytokines in ichthyoses, including NS patients, which also correlated with disease severity (24). These studies did not distinguish between the two major clinical forms of NS patients.

The expression level of IL-36 cytokines was higher in non-lesion NS-SE skin and could contribute to the enhanced IFN signature. Indeed, IL-36 cytokines induce a strong and early expression of STAT1, MX2 and oligoadenylate synthase (OAS) genes in human keratinocytes (46). A recent transcriptomic analysis of blood from pustular and plaque psoriasis patients showed that IL-36 induces a type I IFN response in severe forms of psoriasis (53). Overall, although no marked induction of the IFN pathway was observed in NS blood, it is likely that the IFN pathway contributes to chronic inflammation in NS-SE patient skin.

The description of biological pathways led to considerable progress in the treatment of NS since classical NS treatments, including emollients, calcineurin inhibitors or topical corticosteroids, show a limited benefit (10). The new current treatment of NS mainly relies on intravenous immunoglobulins in children with recurrent infections (13), biotherapy targeting TNF-α, IL-4/IL-13, IL-12/IL-23 or IL-17A, which reduce skin inflammation (63, 26, 64, 65). Recently, the inhibition of the IL-17 axis (27) revealed a duality in the therapeutic response according to the NS endotype, suggesting that other pathways may contribute to NS pathogenesis. Here, we confirm the relevance of blocking IL-17A in both endotypes and the Th2 axis in NS-ILC patients. Our multi-omic approach also revealed a significantly increased IL-36 signature, which has become an attractive therapeutic target with promising results in pustular psoriasis (66, 67). Blocking of IL36 signaling would therefore represent a novel therapeutic strategy for NS, in particular in NS-SE patients.

TABLE 1
Demographic, clinical and molecular characteristics of 13 NS patients included in the study.
Part 1

| | | Age | SPINKS | | NS | Trichorrexis | IgE |
|---|---|---|---|---|---|---|---|
| Patient | Sex | (year) | mutations | Consequences | subtype | invaginate | UI/ml |
| 1 | F | 27 | c.880_882del; | Altered splicing*; | NS-ILC | − | 6217 |
| | | | c.1820 + 2T > A | altered splicing | | | |
| 2 | F | 17 | HMZ: c.1048C > T | p.B350* | NS-SE | + | 282 |
| 1.1 | M | 16 | c.238dup; | p.A217Gfs*19; | NS-ILC | + | 783 |
| | | | c.891C > T | p.C297C (altered | | | |
| | | | | splicing) | | | |
| 1.2 | M | 12 | c.238dup; | p.A217Gfs*19; | NS-ILC | + | 670 |
| | | | c.891C > T | p.C297C (altered | | | |
| | | | | splicing) | | | |
| 4 | F | 21 | c.666 + 1G > A; | Altered splicing; | NS-ILC | − | 519 |
| | | | c.2620_2621insT | p.Y874Lfs*6 | | | |
| 5 | F | 57 | c.2468dup; | p.K824Efs*4 | NS-ILC | + | 7827 |
| | | | c.1351del | p.C451Afs*53 | | | |
| 6 | F | 14 | HMZ: | Retention or 79 bp | NS-ILC | + | 11657 |
| | | | c.2539 − 1501G > C | intronic fragment | | | |
| 7 | F | 18 | c.55 + 1G > A; | Altered splicing; | NS-ILC | − | 14471 |
| | | | c.2015 + 5G > A | altered splicing | | | |
| 8 | F | 34 | HMZ: 55 kb | Partial SPINKS | NS-SE | + | 1259 |
| | | | deletion including | deletion | | | |
| | | | exon 1 to 16 | | | | |
| 9 | M | 28 | c.238dup; | p.A217Gfs*19; | NS-SE | + | 4676 |
| | | | 52 kb deletion | partial SPINKS | | | |
| | | | including exon 5 to 34 | deletion | | | |
| 0.1 | F | 45 | c.283 − 2A > T; | Altered splicing; | NS-ILC | + | 40918 |
| | | | c.2468dup | p.K824Efs*4 | | | |
| 0.2 | F | 43 | c.283 − 2A > T; | Altered splicing; | NS-ILC | + | 15538 |
| | | | c.2468dup | p.K824Efs*4 | | | |
| 0.3 | M | 41 | c.283 − 2A > T; | Altered splicing; | NS-SE | + | 8538 |
| | | | c.2468dup | p.K824Efs*4 | | | |

Part 2

| | Associated | Severity |
|---|---|---|
| Atopy | Manifestations | score |
| Allergic rhinitis, food allergies | Blepharitis, flat warts | 5 |
| Allergic rhinitis, angiodema, multiple | Perinatal dehydration, failure to thrive | 8 |
| food allergies | | |
| Allergic rhinitis, asthma, allergic | | 6 |
| conjunctivitis | | |
| Multiple food allergies, angiodema | | 8 |
| Allergic rhinitis, food allergy (eggs) | Diarrhea in perinatal period | 2 |
| Allergic conjunctivitis, asthma, | Failure to thrive in infancy | 6 |
| angiodema, multiple food allergies | | |
| Allergic rhinitis, allergic | Perinatal dehydration, blepharitis, | 8 |
| conjunctivitis, asthma, multiple food | visual impairment*, obesity*, | |
| allergies | developmental delay^{#} | |
| Allergic rhinitis, angiodema, multiple | Short stature (<2.5 percentile) | 6 |
| food allergies | | |
| — | Perinatal dehydration, blepharitis, | 5 |
| | failure to thrive in infancy, short stature | |
| | (<2.5 percentile) | |
| Allergic rhinitis, allergic | Perinatal dehydration, flat warts, | 8 |
| conjunctivitis, angiodema, multiple | short stature (<2.5 percentile) | |
| food allergies | | |
| Allergic rhinitis, allergic | | 8 |
| conjunctivitis, multiple food allergies | | |
| Allergic rhinitis, allergic | Failure to thrive in infancy, short stature | 8 |
| conjunctivitis, multiple food allergies | (<2.5 percentile), flat warts | |
| Allergic rhinitis, allergic | Perinatal dehydration, failure to thrive | 8 |
| conjunctivitis, angiodema, multiple | in infancy, short stature (<2.5 | |
| food allergies | percentile), papillomatous lesions, flat | |
| | warts | |

ILC, ichthyosis linearis circumflexa; SE, scaly erythroderma. Severity score was established based on the following criteria: skin involvement considering ILC (absent = 0, mild = 1, moderate = 2, severe = 3) or SE (absent = 0, mild = 1, moderate = 2, severe = 3), hair defect SE (absent = 0, mild = 1, moderate = 2, severe = 3) and atopic manifestations SE (absent = 0, mild = 1, moderate = 2, severe = 3).
*This mutation changes the last nucleotide of exon 10 and disrupts the donor splice site of intron 10.
^{#}These features are caused by the association of Bardet-Biedl syndrome confirmed by molecular diagnostic in this patient.

### REFERENCES

Throughout this application, various references describe the state of the art to which this invention pertains. The disclosures of these references are hereby incorporated by reference into the present disclosure.
1. S. Chavanas, C. Bodemer, A. Rochat, D. Hamel-Teillac, M. Ali, A. D. Irvine, J. L. Bonafé, J. Wilkinson, A. Taïeb, Y. Barrandon, J. I. Harper, Y. de Prost, A. Hovnanian, Mutations in SPINK5, encoding a serine protease inhibitor, cause Netherton syndrome, Nat. Genet. 25, 141-142 (2000).
2. E. Bitoun, A. Micheloni, L. Lamant, C. Bonnart, A. Tartaglia-Polcini, C. Cobbold, T. Al Saati, F. Mariotti, J. Mazereeuw-Hautier, F. Boralevi, D. Hohl, J. Harper, C. Bodemer, M. D'Alessio, A. Hovnanian, LEKTI proteolytic processing in human primary keratinocytes, tissue distribution and defective expression in Netherton syndrome, Hum. Mol. Genet. 12, 2417-2430 (2003).
3. A. Ishida-Yamamoto, C. Deraison, C. Bonnart, E. Bitoun, R. Robinson, T. J. O'Brien, K. Wakamatsu, S. Ohtsubo, H. Takahashi, Y. Hashimoto, P. J. C. Dopping-Hepenstal, J. A. McGrath, H. Iizuka, G. Richard, A. Hovnanian, LEKTI is localized in lamellar granules, separated from KLK5 and KLK7, and is secreted in the extracellular spaces of the superficial stratum granulosum, J. Invest. Dermatol. 124, 360-366 (2005).
4. C. Bonnart, C. Deraison, M. Lacroix, Y. Uchida, C. Besson, A. Robin, A. Briot, M. Gonthier, L. Lamant, P. Dubus, B. Monsarrat, A. Hovnanian, Elastase 2 is expressed in human and mouse epidermis and impairs skin barrier function in Netherton syndrome through filaggrin and lipid misprocessing, J. Clin. Invest. 120, 871-882 (2010).
5. P. Descargues, C. Deraison, C. Prost, S. Fraitag, J. Mazereeuw-Hautier, M. D'Alessio, A. Ishida-Yamamoto, C. Bodemer, G. Zambruno, A. Hovnanian, Corneodesmosomal cadherins are preferential targets of stratum corneum trypsin- and chymotrypsin-like hyperactivity in Netherton syndrome, J. Invest. Dermatol. 126, 1622-1632 (2006).
6. C. Deraison, C. Bonnart, F. Lopez, C. Besson, R. Robinson, A. Jayakumar, F. Wagberg, M. Brattsand, J. P. Hachem, G. Leonardsson, A. Hovnanian, LEKTI fragments specifically inhibit KLK5, KLK7, and KLK14 and control desquamation through a pH-dependent interaction, Mol. Biol. Cell 18, 3607-3619 (2007).
7. N. M. Schechter, E.-J. Choi, Z.-M. Wang, Y. Hanakawa, J. R. Stanley, Y. Kang, G. L. Clayman, A. Jayakumar, Inhibition of human kallikreins 5 and 7 by the serine protease inhibitor lympho-epithelial Kazal-type inhibitor (LEKTI), Biol Chem 386, 1173-1184 (2005).
8. P. Fortugno, A. Bresciani, C. Paolini, C. Pazzagli, M. El Hachem, M. D'Alessio, G. Zambruno, Proteolytic activation cascade of the Netherton syndrome-defective protein, LEKTI, in the epidermis: implications for skin homeostasis, J Invest Dermatol 131, 2223-2232 (2011).
9. A. Briot, C. Deraison, M. Lacroix, C. Bonnart, A. Robin, C. Besson, P. Dubus, A. Hovnanian, Kallikrein 5 induces atopic dermatitis-like lesions through PAR2-mediated thymic stromal lymphopoietin expression in Netherton syndrome, J. Exp. Med. 206, 1135-1147 (2009).
10. A. Hovnanian, Netherton syndrome: new advances in the clinic, disease mechanism and treatment, Expert Review of Dermatology 7, 81-92 (2012).
11. A. Hovnanian, Netherton syndrome: skin inflammation and allergy by loss of protease inhibition, Cell Tissue Res. 351, 289-300 (2013).
12. L. Furio, A. Hovnanian, Netherton syndrome: defective kallikrein inhibition in the skin leads to skin inflammation and allergy, Biol. Chem. 395, 945-958 (2014).
13. E. D. Renner, D. Hartl, S. Rylaarsdam, M. L. Young, L. Monaco-Shawver, G. Kleiner, M. L. Markert, E. R. Stichm, B. H. Belohradsky, M. P. Upton, T. R. Torgerson, J. S. Orange, H. D. Ochs, Comèl-Netherton syndrome-defined as primary immunodeficiency, J Allergy Clin Immunol 124, 536-543 (2009).
14. K. Stuvel, J. J. Heeringa, V. A. S. H. Dalm, R. W. J. Meijers, E. van Hoffen, S. A. M. Gerritsen, M. C. van Zelm, S. A. G. M. Pasmans, Comel-Netherton syndrome: a local skin barrier defect in absence of an underlying systemic immunodeficiency, Allergy (2020), doi:10.1111/all.14197.
15. S. Leclerc-Mercier, C. Bodemer, L. Furio, S. Hadj-Rabia, L. de Peufeilhoux, L. Weibel, A.-C. Bursztejn, E. Bourrat, N. Ortonne, T. J. Molina, A. Hovnanian, S. Fraitag, Skin Biopsy in Netherton Syndrome: A Histological Review of a Large Series and New Findings, Am J Dermatopathol 38, 83-91 (2016).
16. A. S. Paller, Y. Renert-Yuval, M. Suprun, H. Esaki, M. Oliva, T. N. Huynh, B. Ungar, N. Kunjravia, R. Friedland, X. Peng, X. Zheng, Y. D. Estrada, J. G. Krueger, K. A. Choate, M. Suárez-Fariñas, E. Guttman-Yassky, An IL-17-dominant immune profile is shared across the major orphan forms of ichthyosis, J. Allergy Clin. Immunol. 139, 152-165 (2017).
17. J. van Smeden, M. Janssens, W. A. Boiten, V. van Drongelen, L. Furio, R. J. Vrecken, A. Hovnanian, J. A. Bouwstra, Intercellular skin barrier lipid composition and organization in Netherton syndrome patients, J. Invest. Dermatol. 134, 1238-1245 (2014).
18. J. van Smeden, H. Al-Khakany, Y. Wang, D. Visscher, N. Stephens, S. Absalah, H. S. Overkleeft, J. M. F. G. Aerts, A. Hovnanian, J. A. Bouwstra, Skin barrier lipid enzyme activity in Netherton patients is associated with protease activity and ceramide abnormalities, J. Lipid Res., jlr.RA120000639 (2020).
19. A. Briot, M. Lacroix, A. Robin, M. Steinhoff, C. Deraison, A. Hovnanian, Par2 inactivation inhibits early production of TSLP, but not cutaneous inflammation, in Netherton syndrome adult mouse model, J. Invest. Dermatol. 130, 2736-2742 (2010).
20. K. Oikonomopoulou, K. K. Hansen, M. Saifeddine, I. Tea, M. Blaber, S. I. Blaber, I. Scarisbrick, P. Andrade-Gordon, G. S. Cottrell, N. W. Bunnett, E. P. Diamandis, M. D. Hollenberg, Proteinase-activated Receptors, Targets for Kallikrein Signaling, J. Biol. Chem. 281, 32095-32112 (2006).
21. K. Stefansson, M. Brattsand, D. Roosterman, C. Kempkes, G. Bocheva, M. Steinhoff, T. Egelrud, Activation of proteinase-activated receptor-2 by human kallikrein-related peptidases, J Invest Dermatol 128, 18-25 (2008).
22. M. R. Williams, L. Cau, Y. Wang, D. Kaul, J. A. Sanford, L. S. Zaramela, S. Khalil, A. M. Butcher, K. Zengler, A. R. Horswill, C. L. Dupont, A. Hovnanian, R. L. Gallo, Interplay of Staphylococcal and Host Proteases Promotes Skin Barrier Disruption in Netherton Syndrome, Cell Rep 30, 2923-2933.e7 (2020).
23. H. Liu, N. K. Archer, C. A. Dillen, Y. Wang, A. G. Ashbaugh, R. V. Ortines, T. Kao, S. K. Lee, S. S. Cai, R. J. Miller, M. C. Marchitto, E. Zhang, D. P. Riggins, R. D. Plaut, S. Stibitz, R. S. Geha, L. S. Miller, Staphylococcus aureus epicutaneous exposure drives skin inflammation via IL-36-mediated T cell responses, Cell Host Microbe 22, 653-666.e5 (2017).
24. K. Malik, H. He, T. N. Huynh, G. Tran, K. Mueller, K. Doytcheva, Y. Renert-Yuval, T. Czarnowicki, S. Magidi, M. Chou, Y. D. Estrada, H.-C. Wen, X. Peng, H. Xu, X. Zheng, J. G. Krueger, A. S. Paller, E. Guttman-Yassky, Ichthyosis molecular fingerprinting shows profound TH17 skewing and a unique barrier genomic signature, J. Allergy Clin. Immunol. 143, 604-618 (2019).
25. T. Czarnowicki, H. He, A. Leonard, K. Malik, S. Magidi, S. Rangel, K. Patel, K. Ramscy, M. Murphrey, T. Song, Y. Estrada, H.-C. Wen, J. G. Krueger, E. Guttman-Yassky, A. S. Paller, The Major Orphan Forms of Ichthyosis Are Characterized by Systemic T-Cell Activation and Th-17/Tc-17/Th-22/Tc-22 Polarization in Blood, J. Invest. Dermatol. 138, 2157-2167 (2018).
26. I. Luchsinger, N. Knöpfel, M. Theiler, M. Bonnet des Claustres, C. Barbieux, A. Schwieger-Briel, C. Brunner, D. Donghi, M. Buettcher, B. Meier-Schiesser, A. Hovnanian, L. Weibel, Secukinumab Therapy for Netherton Syndrome, JAMA Dermatol (2020), doi: 10.1001/jamadermatol.2020.1019.
27. C. Barbieux, M. Bonnet des Claustres, M. de la Brassinne, G. Bricteux, M. Bagot, E. Bourrat, A. Hovnanian, Duality of Netherton syndrome manifestations and response to ixckizumab, J. Am. Acad. Dermatol. (2020), doi: 10.1016/j.jaad.2020.07.054.
28. S. K. Blanchard, N. S. Prose, Successful use of secukinumab in Netherton syndrome, JAAD Case Rep 6, 577-578 (2020).
29. L. C. Tsoi, E. Rodriguez, F. Degenhardt, H. Baurecht, U. Wehkamp, N. Volks, S. Szymczak, W. R. Swindell, M. K. Sarkar, K. Raja, S. Shao, M. Patrick, Y. Gao, R. Uppala, B. E. Percz White, S. Getsios, P. W. Harms, E. Maverakis, J. T. Elder, A. Franke, J. E. Gudjonsson, S. Weidinger, Atopic Dermatitis Is an IL-13-Dominant Disease with Greater Molecular Heterogeneity Compared to Psoriasis, Journal of Investigative Dermatology 139, 1480-1489 (2019).
30. L. C. Tsoi, E. Rodriguez, D. Stölzl, U. Wehkamp, J. Sun, S. Gerdes, M. K. Sarkar, M. Hübenthal, C. Zeng, R. Uppala, X. Xing, F. Thielking, A. C. Billi, W. R. Swindell, A. Shefler, J. Chen, M. T. Patrick, P. W. Harms, J. M. Kahlenberg, B. E. Perez White, E. Maverakis, J. E. Gudjonsson, S. Weidinger, Progression of acute-to-chronic atopic dermatitis is associated with quantitative rather than qualitative changes in cytokine responses, Journal of Allergy and Clinical Immunology (2019), doi: 10.1016/j.jaci.2019.11.047.
31. P. Descargues, C. Deraison, C. Bonnart, M. Kreft, M. Kishibe, A. Ishida-Yamamoto, P. Elias, Y. Barrandon, G. Zambruno, A. Sonnenberg, A. Hovnanian, Spink5-deficient mice mimic Netherton syndrome through degradation of desmoglein 1 by epidermal protease hyperactivity, Nat. Genet. 37, 56-65 (2005).
32. L. Wu, X. Chen, J. Zhao, B. Martin, J. A. Zepp, J. S. Ko, C. Gu, G. Cai, W. Ouyang, G. Sen, G. R. Stark, B. Su, C. M. Vines, C. Tournier, T. A. Hamilton, A. Vidimos, B. Gastman, C. Liu, X. Li, A novel IL-17 signaling pathway controlling keratinocyte proliferation and tumorigenesis via the TRAF4-ERK5 axis, J Exp Med 212, 1571-1587 (2015).
33. Z. Jiang, Y. Liu, C. Li, L. Chang, W. Wang, Z. Wang, X. Gao, B. Ryffel, Y. Wu, Y. Lai, IL-36γ Induced by the TLR3-SLUG-VDR Axis Promotes Wound Healing via REG3A, J Invest Dermatol 137, 2620-2629 (2017).
34. W. Wang, X. Yu, C. Wu, H. Jin, IL-36γ inhibits differentiation and induces inflammation of keratinocyte via Wnt signaling pathway in psoriasis, Int J Med Sci 14, 1002-1007 (2017).
35. C. M. Pfaff, Y. Marquardt, K. Fictkau, J. M. Baron, B. Lüscher, The psoriasis-associated IL-17A induces and cooperates with IL-36 cytokines to control keratinocyte differentiation and function, Scientific Reports 7, 1-13 (2017).
36. L. Mallbris, K. P. O'Brien, A. Hulthen, B. Sandstedt, J. B. Cowland, N. Borregaard, M. Ståhle-Bäckdahl, Neutrophil gelatinase-associated lipocalin is a marker for dysregulated keratinocyte differentiation in human skin: NGAL is a marker for parakeratosis, Experimental Dermatology 11, 584-591 (2002).
37. M. Gschwandtner, M. Mildner, V. Mlitz, F. Gruber, L. Eckhart, T. Werfel, R. Gutzmer, P.

M. Elias, E. Tschachler, Histamine suppresses epidermal keratinocyte differentiation and impairs skin barrier function in a human skin model, Allergy 68, 37-47 (2013).
38. D. R. Bielenberg, M. F. McCarty, C. D. Bucana, S. H. Yuspa, D. Morgan, J. M. Arbeit, L. M. Ellis, K. R. Cleary, I. J. Fidler, Expression of Interferon-is Associated with Growth Arrest of Murine and Human Epidermal Cells, Journal of Investigative Dermatology 112, 802-809 (1999).
39. H. Blumberg, H. Dinh, E. S. Trueblood, J. Pretorius, D. Kugler, N. Weng, S. T. Kanaly, J. E. Towne, C. R. Willis, M. K. Kuechle, J. E. Sims, J. J. Peschon, Opposing activities of two novel members of the IL-1 ligand family regulate skin inflammation, J Exp Med 204, 2603-2614 (2007).
40. Y. Carrier, H.-L. Ma, H. E. Ramon, L. Napierata, C. Small, M. O'Toole, D. A. Young, L. A. Fouser, C. Nickerson-Nutter, M. Collins, K. Dunussi-Joannopoulos, Q. G. Medley, Inter-regulation of Th17 cytokines and the IL-36 cytokines in vitro and in vivo: implications in psoriasis pathogenesis, J. Invest. Dermatol. 131, 2428-2437 (2011).
41. H.-H. Chi, K.-F. Hua, Y.-C. Lin, C.-L. Chu, C.-Y. Hsich, Y.-J. Hsu, S.-M. Ka, Y.-L. Tsai, F.-C. Liu, A. Chen, IL-36 Signaling Facilitates Activation of the NLRP3 Inflammasome and IL-23/IL-17 Axis in Renal Inflammation and Fibrosis, J. Am. Soc. Nephrol. 28, 2022-2037 (2017).
42. C. Bridgewood, G. W. Fearnley, A. Berekmeri, P. Laws, T. Macleod, S. Ponnambalam, M. Stacey, A. Graham, M. Wittmann, IL-36γ Is a Strong Inducer of IL-23 in Psoriatic Cells and Activates Angiogenesis, Front Immunol 9, 200 (2018).
43. S. K. Mahil, M. Catapano, P. Di Meglio, N. Dand, H. Ahlfors, I. M. Carr, C. H. Smith, R. C. Trembath, M. Peakman, J. Wright, F. D. Ciccarelli, J. N. Barker, F. Capon, An analysis of IL-36 signature genes and individuals with IL1RL2 knockout mutations validates IL-36 as a psoriasis therapeutic target, Sci Transl Med 9 (2017), doi: 10.1126/scitranslmed.aan2514.
44. B. Germán, R. Wei, P. Hener, C. Martins, T. Ye, C. Gottwick, J. Yang, J. Seneschal, K. Boniface, M. Li, Disrupting the IL-36 and IL-23/IL-17 loop underlies the efficacy of calcipotriol and corticosteroid therapy for psoriasis, JCI Insight 4 (2019), doi: 10.1172/jci.insight.123390.
45. Y. E. Hernández-Santana, G. Leon, D. St Leger, P. G. Fallon, P. T. Walsh, Keratinocyte interleukin-36 receptor expression orchestrates psoriasiform inflammation in mice, Life Sci Alliance 3 (2020), doi: 10.26508/lsa.201900586.
46. W. R. Swindell, M. A. Beamer, M. K. Sarkar, S. Loftus, J. Fullmer, X. Xing, N. L. Ward, L. C. Tsoi, M. J. Kahlenberg, Y. Liang, J. E. Gudjonsson, RNA-Seq Analysis of IL-1B and IL-36 Responses in Epidermal Keratinocytes Identifies a Shared MyD88-Dependent Gene Signature, Front Immunol 9, 80 (2018).
47. A. Müller, A. Hennig, S. Lorscheid, P. Grondona, K. Schulze-Osthoff, S. Hailfinger, D. Kramer, IκBζ is a key transcriptional regulator of IL-36-driven psoriasis-related gene expression in keratinocytes, PNAS 115, 10088-10093 (2018).
48. A. M. D'Erme, D. Wilsmann-Theis, J. Wagenpfeil, M. Hölzel, S. Ferring-Schmitt, S. Sternberg, M. Wittmann, B. Peters, A. Bosio, T. Bieber, J. Wenzel, IL-36γ (IL-1F9) is a biomarker for psoriasis skin lesions, J. Invest. Dermatol. 135, 1025-1032 (2015).
49. F. Kolbinger, C. Loesche, M.-A. Valentin, X. Jiang, Y. Cheng, P. Jarvis, T. Peters, C. Calonder, G. Bruin, F. Polus, B. Aigner, D. M. Lee, M. Bodenlenz, F. Sinner, T. R. Picber, D. D. Patel, B-Defensin 2 is a responsive biomarker of IL-17A-driven skin pathology in patients with psoriasis, Journal of Allergy and Clinical Immunology 139, 923-932.e8 (2017).
50. J. Giang, M. A. J. Scclen, M. B. A. van Doorn, R. Rissmann, E. P. Prens, J. Damman, Complement Activation in Inflammatory Skin Diseases, Front Immunol 9 (2018), doi: 10.3389/fimmu.2018.00639.
51. C. Giacomassi, N. Buang, G. S. Ling, G. Crawford, H. T. Cook, D. Scott, F. Dazzi, J. Strid, M. Botto, Complement C3 Exacerbates Imiquimod-Induced Skin Inflammation and Psoriasiform Dermatitis, J. Invest. Dermatol. 137, 760-763 (2017).
52. F. O. Nestle, C. Conrad, A. Tun-Kyi, B. Homey, M. Gombert, O. Boyman, G. Burg, Y.-J. Liu, M. Gillict, Plasmacytoid predendritic cells initiate psoriasis through interferon-a production, J Exp Med 202, 135-143 (2005).
53. M. Catapano, M. Vergnano, M. Romano, S. K. Mahil, S.-E. Choon, A. D. Burden, H. S. Young, I. M. Carr, H. J. Lachmann, G. Lombardi, C. H. Smith, F. D. Ciccarelli, J. N. Barker, F. Capon, IL-36 Promotes Systemic IFN-I Responses in Severe Forms of Psoriasis, Journal of Investigative Dermatology 140, 816-826.c3 (2020).
54. R. Lande, J. Gregorio, V. Facchinetti, B. Chatterjee, Y.-H. Wang, B. Homey, W. Cao, Y.-H. Wang, B. Su, F. O. Nestle, T. Zal, I. Mellman, J.-M. Schröder, Y.-J. Liu, M. Gilliet, Plasmacytoid dendritic cells sense self-DNA coupled with antimicrobial peptide, Nature 449, 564-569 (2007).
55. C. Conrad, M. Gilliet, Type I IFNs at the Interface between Cutaneous Immunity and Epidermal Remodeling, Journal of Investigative Dermatology 132, 1759-1762 (2012).
56. L. Zhang, Typel Interferons Potential Initiating Factors Linking Skin Wounds With Psoriasis Pathogenesis, Front. Immunol. 10 (2019), doi: 10.3389/fimmu.2019.01440.
57. K. Hannula-Jouppi, S.-L. Laasanen, H. Heikkilä, M. Tuomiranta, M.-L. Tuomi, S. Hilvo, N. Kluger, S. Kivirikko, A. Hovnanian, S. Mäkinen-Kiljunen, A. Ranki, IgE allergen component-based profiling and atopic manifestations in patients with Netherton syndrome, J. Allergy Clin. Immunol. 134, 985-988 (2014).
58. L. Chen, S. Lin, R. Agha-Majzoub, L. Overbergh, C. Mathieu, L. S. Chan, CCL27 is a critical factor for the development of atopic dermatitis in the keratin-14 IL-4 transgenic mouse model, Int. Immunol. 18, 1233-1242 (2006).
59. S. Schra, W. Yao, E. T. Nguyen, N. L. Glosson-Byers, N. Akhtar, B. Zhou, M. H. Kaplan, TH9 cells are required for tissue mast cell accumulation during allergic inflammation, J. Allergy Clin. Immunol. 136, 433-440.e1 (2015).
60. A. Pajulas, M. H. Kaplan, The role of IL-9 secreting CD4+T helper cells in promoting mast cell expansion in pulmonary models of inflammation, The Journal of Immunology 204, 65.22-65.22 (2020).
61. A. Harusato, H. Abo, V. Le Ngo, S. W. Yi, K. Mitsutake, S. Osuka, J. E. Kohlmeier, J.-D. Li, A. T. Gewirtz, A. Nusrat, T. L. Denning, IL-36γ signaling controls the induced regulatory T cell-TH9 cell balance via NFκB activation and STAT transcription factors, Mucosal Immunol 10, 1455-1467 (2017).
62. M. T. Wong, J. J. Ye, M. N. Alonso, A. Landrigan, R. K. Cheung, E. Engleman, P. J. Utz, Regulation of human Th9 differentiation by type I interferons and IL-21, Immunol Cell Biol 88, 624-631 (2010).
63. L. Fontao, E. Laffitte, A. Briot, G. Kaya, P. Roux-Lombard, S. Fraitag, A. A. Hovnanian, J.-H. Saurat, Infliximab infusions for Netherton syndrome: sustained clinical improvement correlates with a reduction of thymic stromal lymphopoietin levels in the skin, J. Invest. Dermatol. 131, 1947-1950 (2011).
64. A. B. Steuer, D. E. Cohen, Treatment of Netherton Syndrome With Dupilumab, JAMA Dermatol (2020), doi: 10.1001/jamadermatol.2019.4608.
65. S. Volc, L. Maier, A. Gritsch, M. C. Aichelburg, B. Volc-Platzer, Successful treatment of Netherton syndrome with ustekinumab in a 15-year-old girl, Br. J. Dermatol. (2020), doi: 10.1111/bjd.18892.
66. V. Todorović, Z. Su, C. B. Putman, S. J. Kakavas, K. M. Salte, H. A. McDonald, J. B. Wetter, S. E. Paulsboe, Q. Sun, C. E. Gerstein, L. Medina, B. Sielaff, R. Sadhukhan, H. Stockmann, P. L. Richardson, W. Qiu, M. A. Argiriadi, R. F. Henry, J. M. Herold, J. B. Shotwell, S. P. McGaraughty, P. Honore, S. M. Gopalakrishnan, C. C. Sun, V. E. Scott, Small Molecule IL-36γ Antagonist as a Novel Therapeutic Approach for Plaque Psoriasis, Scientific Reports 9, 1-15 (2019).
67. H. Bachelez, S.-E. Choon, S. Marrakchi, A. D. Burden, T.-F. Tsai, A. Morita, H. Turki, D.

B. Hall, M. Shear, P. Baum, S. J. Padula, C. Thoma, Inhibition of the Interleukin-36 Pathway for the Treatment of Generalized Pustular Psoriasis, N. Engl. J. Med. 380, 981-983 (2019).
68. M. D. Howell, F. I. Kuo, P. A. Smith, Targeting the Janus Kinase Family in Autoimmune Skin Diseases, Front. Immunol. 10, 2342 (2019).
69. F. Solimani, K. Meier, K. Ghoreschi, Emerging Topical and Systemic JAK Inhibitors in Dermatology, Front. Immunol. 10, 2847 (2019).
70. T. P. Singh, M. P. Schon, K. Wallbrecht, A. Gruber-Wackernagel, X.-J. Wang, P. Wolf, A. Zernecke, Ed. Involvement of IL-9 in Th17-Associated Inflammation and Angiogenesis of Psoriasis, PLOS ONE 8, e51752 (2013).

1. A method of treating Netherton syndrome in a patient in need thereof comprising administering to the patient a therapeutically effective amount of an IL-36 inhibitor.

2. The method of claim 1 wherein the patient retains a scaly erythrodermic phenotype (SE).

3. The method of claim 1 wherein the IL-36 inhibitor is selected from the group consisting of an antibody directed against the IL-36 cytokine and an antibody directed against the IL-36 receptor.

4. The method of claim 3 wherein the antibody binds to the extracellular domain of IL1RL2.

5. The method of claim 4 wherein the antibody binds to the amino acid sequence that ranges from the amino acid residue at position 20 to the amino acid residue 335 in SEQ ID NO:1.

6. The method of claim 5 wherein the antibody binds to a conformational epitope that comprises at least one amino acid sequences selected from the group consisting of MKNEIL (SEQ ID NO:2), EKHWCDTSIGGLPNL (SEQ ID NO:3), YKQILHL (SEQ ID NO:4), IKGERF (SEQ ID NO: 5 and QAILTHSGKQ (SEQ ID NO:6).

7. The method of claim 1 wherein the IL-36 inhibitor is an inhibitor of an IL-36 cytokine expression or of an IL-36 receptor expression.

8. The method of claim 1 wherein the IL-36 inhibitor is administered to the patient in a topical formulation.

9. The method of claim 3, wherein the antibody specifically binds IL1RL2 or IL1RAP or the dimeric complex formed thereby.

10. The method of claim 7, wherein the IL-36 inhibitor is an inhibitor of IL1RL2 or IL1RAP expression.
